(19) United States
(12) Patent Application Publication
(10) Pub. No.: US 2022/0213430 A1
(43) Pub. Date: Jul. 7, 2022

(54) METHODS OF TISSUE PROCESSING

(71) Applicant: VIADIGM LLC, Birmingham, AL (US)

(72) Inventor: Jeremy B. Vines, Birmingham, AL (US)

(73) Assignee: VIADIGM LLC, Birmingham, AL (US)

(21) Appl. No.: 17/605,521

(22) PCT Filed: Apr. 24, 2020
(86) PCT No.: PCT/US2020/029742

§ 371 Date: Oct. 21, 2021

Related U.S. Application Data
(60) Provisional application No. 62/838,245, filed on Apr. 24, 2019.

(51) Int. Cl. C12N 1/06; A61K 35/50; C12N 5/073; C12N 1/04

(52) U.S. Cl. CPC C12N 1/066; C12N 1/04; C12N 5/0605; A61K 35/50

(57) ABSTRACT

The present technology provides a method for the effective participation of tissue, for example, amniotic, chorionic, umbilical cord, or a combination thereof to create a flowable suspension for therapeutic purposes. In contrast to other technologies, the present technology does not require cryogenic milling or pre-freezing to harden the tissue for effective particulation and is further capable of preserving the viability of endogenous cell types in contrast to previous methodologies.

### CROSS REFERENCE

This application claims priority to U.S. Provisional Application Ser. No. 62/838,245 filed Apr. 24, 2019, incorporated by reference herein in its entirety.

### BACKGROUND OF THE INVENTION

### Field of the Invention

Methods of preparing tissue material are provided. Compositions and kits are also provided.

### State of the Art

Placental tissue contains many beneficial properties for therapeutic applications. The placenta is a unique fetomaternal organ whose main function is to ensure the uninhibited coexistence of a mother and fetus such that the growth and development of the fetus may proceed without difficulty. In this regard, the placenta, and its associated membranes support the fetus by removing metabolic products and preventing immunological rejection of the fetus by the mother. The placenta is comprised of both embryo, and maternal-derived tissues including the amniotic and chorionic membranes and the umbilical cord.

Placental tissues may provide substantial therapeutic benefit due to their immunomodulatory, bacteriostatic, and regenerative properties (King et al., Expression of natural antimicrobials by human placenta and fetal membranes. Placenta. 2007; 28:161-9.; Veenstra van Nieuwenhoven et al., The immunology of successful pregnancy. Hum Reprod Update. 2003; 9:347-57; Liu et al., Human placenta-derived adherent cells induce tolerogenic immune responses. Clin Transl Immunology. 2014; 3:e14.; Whitley G S and Cartwright J E. Trophoblast-mediated spiral artery remodelling: a role for apoptosis. J Anat. 2009; 215:21-6.). These tissues are populated with a variety of cellular populations, for example, amniotic membrane derived mesenchymal stem cells and epithelial cells, chorionic derived mesenchymal stem cells, umbilical cord blood stem cells, etc. (PMID: 21786036), and growth factors such as TIMP-1, TIMP-2, TIMP-3, ANG, VEGF, IGF-1, IGF-2, IGF-3, EGF, bFGF, HGF and TGF-B1, among others (Litwiniuk et al., Amount and distribution of selected biologically active factors in amniotic membrane depends on the part of amnion and mode of childbirth. Can we predict properties of amnion dressing? A proof-of-concept study. Cent Eur J Immunol. 2018; 43:97-102.; Lei et al., Identification of Extracellular Matrix Components and Biological Factors in Micronized Dehydrated Human Amnion/Chorion Membrane. Adv Wound Care (New Rochelle). 2017; 6:43-53.). In addition, placental tissues often contain bacteriostatic compounds such as beta defensins (King et al., Expression of natural antimicrobials by human placenta and fetal membranes. Placenta. 2007; 28:161-9.; Stachon et al., [Growth Factors and Interleukins in Amniotic Membrane Tissue Homogenate]. Klin Monbl Augenheilkd. 2015:232:858-62.).

Placental tissues are evolutionarily designed to protect the fetus from maternal immunological aggression (Pogozhykh et al., Placenta and Placental Derivatives in Regenerative Therapies: Experimental Studies, History, and Prospects. Stem Cells Int. 2018; 2018:4837930.). For example, trophoblastic cells have little to no expression of MHC Class I (HLA-A, HLA-B, HLA-C) markers while other cells found within the placenta and its associated tissues exhibit much lower expression of these markers (Chen et al., Immunologic regulation in pregnancy: from mechanism to therapeutic strategy for immunomodulation. Clin Dev Immunol. 2012; 2012:258391.; Kanellopoulos-Langevin et al., Tolerance of the fetus by the maternal immune system: role of inflammatory mediators at the feto-maternal interface. Reprod Biol Endocrinol. 2003; 1:121.). Additionally, various placental cells express the non-traditional MHC marker, HLA-G, which is known to inhibit the proliferation of T-lymphocytes by interacting with the NKR2B4 receptor, and inhibiting the migration of natural killer cells. (Veenstra van Nieuwenhoven et al. The immunology of successful pregnancy. Hum Reprod Update. 2003; 9:347-57.; Regnault et al., Placental development in normal and compromised pregnancies—a review. Placenta. 2002; 23 Suppl A:S119-29.; Askelund K J and Chamley L W. Trophoblast deportation part I: review of the evidence demonstrating trophoblast shedding and deportation during human pregnancy. Placenta. 2011; 32:716-23.). Other immunomodulatory features of cells within the placenta include the expression of apoptosis-inducing ligands FasL and TRAIL, which exert their function on immunological cell types (PMID: 19215319). Furthermore, pregnancy can be accompanied by the remission of many autoimmune diseases (Veenstra van Nieuwenhoven et al., The immunology of successful pregnancy. Hum Reprod Update. 2003; 9:347-57.; Liu et al., Human placenta-derived adherent cells induce tolerogenic immune responses. Clin Transl Immunology. 2014; 3:e14.). Of benefit to wound healing applications is the fact that the human placenta and its associated membranes express natural antimicrobial molecules such as beta defensins and elafin (King et al., Expression of natural antimicrobials by human placenta and fetal membranes. Placenta. 2007; 28:161-9.). In particular, the placental trophoblast and chorionic membrane are the primary layers providing anti-bacterial function within the placenta.

These factors make placental tissues useful in a wide variety of regenerative medicine applications including ocular, orthopedic, adhesion barrier, and wound healing applications, and placental tissues are increasingly used for regenerative medicine applications such as bony fusions, articular cartilage repair, and wound healing. For therapeutic purposes, it is necessary to process the tissue into a flowable, particulated/micronized form. However, due to the soft and ductile nature of placental tissues, it is often difficult to particulate tissue into a flowable form using normal mechanical means without first freezing the tissue to make it brittle enough for processing. Many existing technologies employ the utilization of methods such as cryogenic bead/ball mills (US 2015/0086573 A1) or other methods such as mortar/pestle and dounce homogenizers (WO 2017/004460 A1) that are harsh and significant decrease in the viability of cellular components. For example, it is well known that cryopreservation results in a loss of viability around 30% on average (Svalgaard et al., Pentaisomaltose, an Alternative to DMSO. Engraftment of Cryopreserved Human CD34(+) Cells in Immunodeficient NSG Mice. Cell Transplant. 2018; 27:1407-1412.; Svalgaard et al., Low-molecular-weight carbohydrate Pentaisomaltose may replace dimethyl sulfoxide as a safer cryoprotectant for cryopreservation of peripheral blood stem cells. Transfusion. 2016; 56:1088-95.). Homogenization after initial tissue freezing using dounce homogenizers, French press, French pressure cells, gentle vortex bead beating, or a mortar and pestle are aggressive and result in a loss of viability of the tissue. Furthermore, the process of flash freezing and thawing multiple times influences the nature of the protein composition. It has been shown that the freezing rate, as well as the amount of multiple freeze thaw cycles, negatively influence protein activity depending on the protein structure and sensitivity (Cao et al., Effect of freezing and thawing rates on denaturation of proteins in aqueous solutions. Biotechnol Bioeng. 2003; 82:684-90.; PGM A M, Moriel P and Arthington J D. Effects of storage temperature and repeated freeze-thaw cycles on stability of bovine plasma concentrations of haptoglobin and ceruloplasmin. J Vet Diagn Invest. 2017; 29:738-740.; Gislefoss et al., Effect of multiple freeze-thaw cycles on selected biochemical serum components. Clin Chem Lab Med. 2017; 55:967-973.).

There is a need for a method for quick particulation of tissue resulting in viable cellular material for therapeutic purposes.

### SUMMARY OF THE INVENTION

The present technology provides a facile and gentle method for the effective particulation of placental tissues in contrast to other methodologies that require aggressive grinding and thermal cycling/freezing of tissue to achieve flowable, particulated tissue suspensions. Various embodiments of the present methodology may comprise use of blunt mincing using a variety of methods, placing minced tissue into the loading chamber of a sieve and rotor based mill loaded with frozen isotonic solution for fine particulation, rinsing and antibiotic treatment steps, and re-suspension for final cryopreservation using controlled rate freezing.

In an aspect, this disclosure provides a method of producing a particulated tissue, the method comprising:
(a) obtaining a tissue sample;
(b) mincing the tissue sample to produce a minced tissue;
(c) loading an isotonic solution into a processing chamber;
(d) adding the minced tissue to the isotonic solution in the processing chamber,
(e) milling the minced tissue in the processing chamber into the particulated tissue; and
(f) collecting the particulated tissue.

In another aspect, this disclosure provides a method of producing a particulated tissue, the method comprising:
(a) obtaining a tissue sample;
(b) mincing the tissue sample to produce a minced tissue;
(c) loading an intermediary material into a processing chamber;
(d) adding the minced tissue to the intermediary material in the processing chamber,
(e) milling the minced tissue in the processing chamber into the particulated tissue; and
(f) collecting the particulated tissue.

In certain embodiments of the methods disclosed herein, wherein the tissue sample is one or more of placental tissue, amniotic membrane, chorionic membrane, or umbilical cord tissue. In some embodiments, the tissue sample is minced into pieces from about 0.10 cm² to about 1.0 cm²in size. In an embodiment, the tissue sample is minced into pieces from about 0.2 cm² to about 0.50 cm²in size.

In certain embodiments of the methods disclosed herein, the isotonic solution is frozen into a frozen isotonic solution. In some embodiments, the frozen isotonic solution is about 0.30 cm² to about 2.5 cm²in size. In certain embodiments of the methods disclosed herein, the processing chamber is a rotor mill, and the rotor mill comprises a sieve with a pore size between about 200 μm and about 1 mm. In an embodiment, the pore size of the sieve is about 500 μm.

In certain embodiments of the methods disclosed herein, the minced tissue and the isotonic solution or the intermediary material are milled in the rotor mill at about 3,000 rpm to about 18,000 rpm. In an embodiment, the minced tissue and the isotonic solution or the intermediary material are milled in the rotor mill at about 6,000 rpm.

In certain embodiments of the methods disclosed herein, the minced tissue and the isotonic solution or the intermediary material are milled in the rotor mill for about 5 seconds to about 60 seconds. In an embodiment, the minced tissue and the isotonic solution or the intermediary material are milled in the rotor mill for about 10 seconds.

In certain embodiments of the methods disclosed herein, comprise purging the processing chamber with the isotonic solution to collect residual particulated tissue. In some embodiments, the methods further comprise purging the processing chamber with an additional isotonic solution to collect residual particulated tissue, wherein the additional isotonic solution is a different isotonic solution than the isotonic solution used in step (c).

In another aspect, the disclosure provides compositions comprising the particulated tissue prepared by any of the methods disclosed herein.

In yet another aspect, the disclosure provides a kit comprising the composition as disclosed herein in a pharmaceutically acceptable carrier, and a label or instructions for administration of the composition to treat a subject.

In another aspect, the disclosure provides methods of treating a disorder and/or a disease in a subject, wherein the method comprises administering to the subject a therapeutically effective amount of any the compositions as disclosed herein.

In some embodiments, the disorder and/or disease is selected from the group consisting of: an ocular disease (for example, refractive errors, macular degeneration, age-related macular degeneration, cataracts, or uveitis), wound healing (for example, following a surgery, a burn, a trauma or tissue damage), arthritis (for example, osteoarthritis, inflammatory arthritis, rheumatoid arthritis, ankylosing spondylitis, psoriatic arthritis, lupus and/or degeneration and/or inflammation of connective tissues such as cartilage, articular cartilage defect, meniscal damage, osteochondritis dissecans, or aseptic necrosis), an immune-related disease, graft-versus-host disease, and an angiogenesis-related disease.

Furthermore, provided herein are methods of regenerating or repairing a tissue and/or an organ in a subject in need thereof, wherein the methods comprise administering to the subject a therapeutically effective amount of any the compositions as disclosed herein to regenerate or repair the tissue or organ in the subject. In some embodiments, the tissue or organ may be part of the subject's skin, joints, bones, cartilage, respiratory tract, gastrointestinal tract, cardiovascular system, liver, pancreas, bone marrow, or central nervous system. In certain embodiments, administration of the therapeutically effective amount of any the compositions may be topical, transdermal, mucosal, sub-mucosal, muscular, sub-muscular, by inhalation, parenteral, or intravenous administration.

As used herein, the terms “subject” or “patient” to be treated refer to an animal, preferably to a mammal, even more preferably to a human, including an adult, a child, and a human at the prenatal stage. In some embodiments, a subject can refer to a non-human mammal. In certain embodiments, a subject is a human subject.

As used herein, the term “treatment”, “treat” or “treating” refers to any act intended to ameliorate the health status of patients such as therapy, prevention, prophylaxis and retardation of the disorder and/or disease. In certain embodiments, such term refers to the amelioration or eradication of a disease or symptoms associated with a disease as disclosed herein. In other embodiments, this term refers to minimizing the spread or worsening of the disease resulting from the administration of one or more therapeutic agents to a subject with such a disease.

The term “therapeutically effective amount” refers to an amount of any of the compositions as disclosed herein that is administered to a subject that is sufficient to constitute a treatment of the disorder and/or disease as disclosed above. In some embodiments, this term may refer to the amount of the composition necessary to repair and/or regenerate the tissue(s) or organ(s) of the subject being treated.

The compositions for use in the methods as disclosed herein are typically “pharmaceutically acceptable”. The term “pharmaceutically acceptable” as used herein refers to compositions comprising various components, such as, for example, carriers, diluents, preservatives, and/or cryopreservatives, and such components are typically suitable for administration to a human subject, as well as to a non-human mammalian subject.

In certain embodiments, the compositions can be administered one or more times in order to effectively treat the subject. A decision as to how many doses (and when to administer the dose(s)) will typically be made by an individual who is supervising the subject's treatment, such as a treating physician, doctor, veterinarian, or nurse. A combination of factors will typically be taken into account in making such a decision. For example, factors taken in to account may include a timescale of appropriateness as assessed on the basis of past experience, either with the individual subject patient's condition or others with similar conditions, or on the degree of debility of the subject, or on the degree of pain experienced by the subject or may be based on a test independent of the subject's own assessment.

### BRIEF DESCRIPTION OF THE DRAWINGS

FIG. 1: Schematic of typical sieved rotor-mill configuration illustrating collection pan, sieve, and toothed rotor to be used for particulation of tissues (top), with sterile frozen isotonic solution and minced tissue pre-loaded into rotor (bottom).

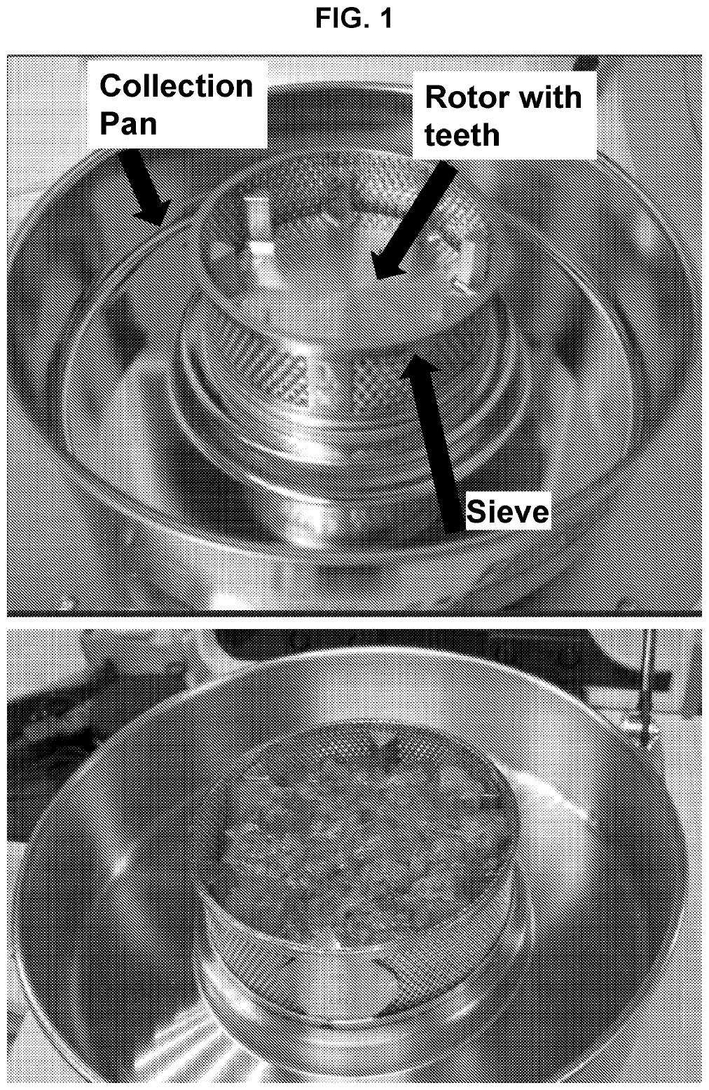

FIG. 2: Processing Picture 1: Mincing of tissue before loading into rotor mill.

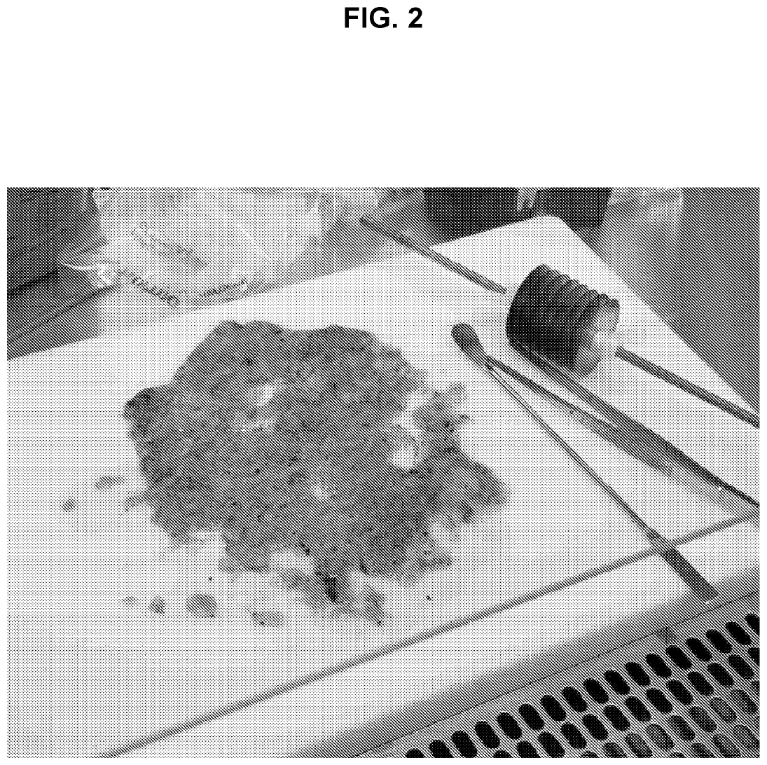

FIG. 3: Processing Picture 2: Post-processed, particulated tissue in collection pan.

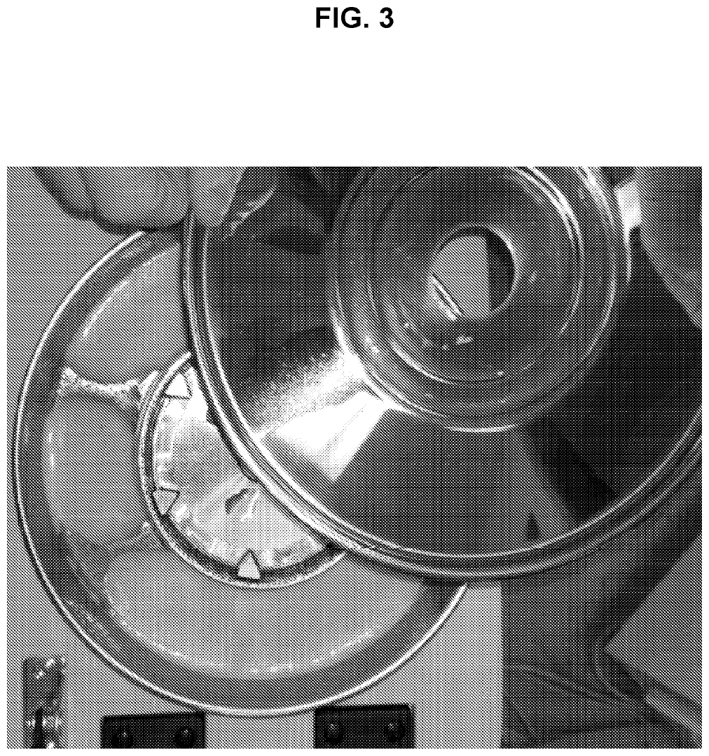

FIG. 4: AlamarBlue™ viability measurement of particulated suspension post-processing and influence of rotor mill loading conditions on post-processed tissue viability.

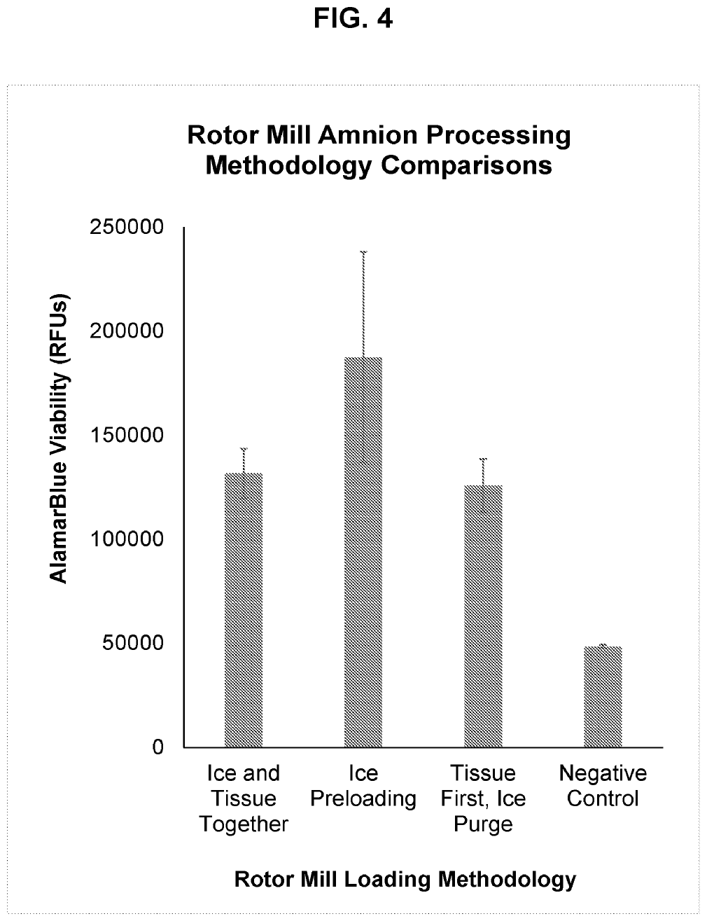

FIG. 5: AlamarBlue™ viability measurement of particulated suspension post-processing and influence of needle gauge on post-injection viability.

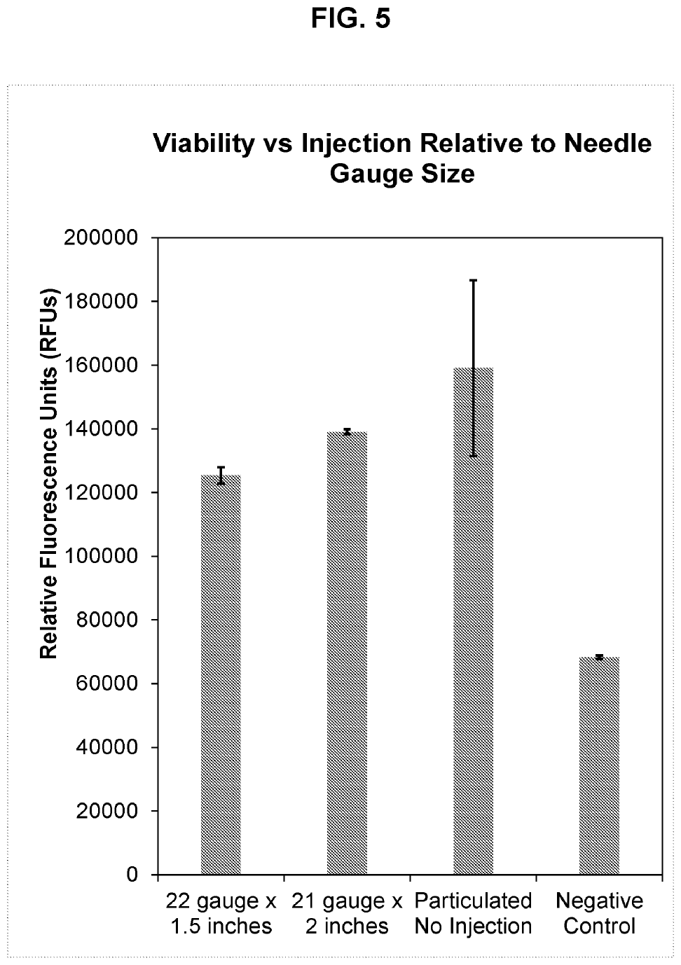

### DETAILED DESCRIPTION

A majority of the placental tissues intended for clinical use are processed as minimally manipulated allografts classified under the Food and Drug Administration's (FDA's) criterion of minimal manipulation under 21 CFR 1271.10(a)(1). This FDA classification refers to preparations of human cells, tissues, and cell/tissue based products (HCT/P 361) intended for implantation, transplantation, infusion, or transfer to a human recipient. To maintain status as a minimally manipulated tissue, the processing should not alter the relevant biological characteristics of the cells and tissues so that the utility of the tissue for replacement, repair, or reconstruction is not reduced.

The exemplary methods described for the present technology are designed to process tissues in a way that qualifies it as minimally manipulated by avoiding substantial morphological changes to cells, growth factors, and extracellular matrix proteins. In this regard, aggressive processing methodologies such as lyophilizing, heavy grinding/smashing, macerating, flash freezing, thermal cycling via multiple freeze-thaw cycles, blending, and sonicating are not necessary. The methods may be used to particulate any tissue. In particular, the methods may be used to particulate amniotic membrane, chorionic membrane, umbilical cord tissue, or a combination of these tissues.

All publications, patents and patent applications cited herein are hereby expressly incorporated by reference for all purposes.

Before describing the present invention in detail, a number of terms will be defined. As used herein, the singular forms “a,” “an,” and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

In an aspect, this disclosure provides a method of producing a particulated tissue, the method comprising:
(a) obtaining a tissue sample;
(b) mincing the tissue sample to produce a minced tissue;
(c) loading an isotonic solution into a processing chamber;
(d) adding the minced tissue to the isotonic solution in the processing chamber,
(e) milling the minced tissue in the processing chamber into the particulated tissue; and
(f) collecting the particulated tissue.

In another aspect, this disclosure provides a method of producing a particulated tissue, the method comprising:
(a) obtaining a tissue sample;
(b) mincing the tissue sample to produce a minced tissue;
(c) loading an intermediary material into a processing chamber;
(d) adding the minced tissue to the intermediary material in the processing chamber,
(e) milling the minced tissue in the processing chamber into the particulated tissue; and
(f) collecting the particulated tissue.

The methods as disclosed herein can be used to process any tissue. Tissue can refer to any soft tissue that connects, supports, or surrounds other structures and organs of the body (not being hard tissue such as bone). Tissues that can be processed by the methods disclosed herein can include, but are not limited to, tendons, ligaments, fascia, skin, fibrous tissues, fat, and synovial membranes (which are connective tissue), and muscles, nerves and blood vessels (which are not connective tissue). In certain embodiments, the tissue comprises placental membrane and amnion tissue (which can include amniotic fluid cells, placental membrane, amnion tissue or combinations thereof). Placental membrane and amnion tissue refer to one or more layers of the placental membrane, and can include tissue components of the placental organ including, but not limited to, the placental globe, the umbilical cord, the chorionic membrane, the amniotic membrane, and other gelatins, cells and extracellular material. In certain embodiments, placental membrane or amnion tissue may refer to a placental membrane comprising both the amniotic and chorionic layers.

In some embodiments, placental membrane or amnion tissue may refer to a placental membrane in which the chorion has been removed. In other embodiments, placental membrane or amnion tissue may refer to a placental membrane in which the epithelial layer has been removed. The tissues processed by the methods disclosed herein can be obtained from any animal. For example, the tissues can be obtained from animals including, but not limited to, humans, primates, mammals, equines, cattle, swine, sheep, poultry, cats and dogs. In certain embodiments, the tissue is obtained from humans.

In some embodiments, the tissue is placental membrane tissue comprising both the amniotic and chorionic layers and is obtained from consenting donors in accordance with the Current Good Tissue Practice guidelines promulgated by the U.S. Food and Drug Administration and the American Association of Tissue Banks (AATB). For example, donors typically undergo standard STD and other infectious disease screening as part of normal pregnancy. Donors are also asked to submit a social history survey documenting risky behaviors, including behaviors associated with Zika virus. In certain embodiments, the tissue is obtained soon after the birth of a human infant via an elective Cesarean section delivery, the intact placenta is retrieved, and the placental membrane is dissected from the placenta. Afterwards, the placental membrane is cleaned of residual blood, placed in a bath of sterile solution, stored on ice and shipped for processing.

In certain embodiments of the methods disclosed herein, the tissue to be processed is rinsed to remove any remaining blood dots. For example, bloody chorion surfaces can be gently rubbed to remove clots. In some embodiments, a cell scraper to can be used to gently remove clots from the surface (for example, smaller, more persistent dots). In certain embodiments, the tissue may need to be rinsed multiple times in order to remove clots. In some embodiments, the tissue is rinsed in ACK lysing buffer under conditions and for a time required to remove any clots.

In some embodiments of the methods as disclosed herein, the tissue is contacted with or rinsed in an antibiotic rinse. The antibiotic rinse may include, but is not limited to, the antibiotics: amikacin, aminoglycosides, amoxicillin, ampicillin, ansamycins, arsphenamine, azithromycin, azlocillin, aztreonam, bacitracin, capreomycin, carbacephem, carbapenems, carbenicillin, cefaclor, cefadroxil, cefalexin, cefalotin, cefamandole, cefazolin, cefdinir, cefditoren, cefepime, cefixime, cefoperazone, cefotaxime, cefoxitin, cefpodoxime, cefprozil, ceftaroline fosamil, ceftazidime, ceftibuten, ceftizoxime, ceftobiprole, ceftriaxone, cefuroxime, chloramphenicol, ciprofloxacin, clarithromycin, clindamycin, clofazimine, cloxacillin, colistin, cycloserine, dapsone, daptomycin, demecdocycline, dicloxacillin, dirithromycin, doripenem, doxycycline, enoxacin, ertapenem, erythromycin, ethambutol, ethionamide, flucoxacillin, fosfomycin, furazolidone, fusidic acid, gatifloxacin, geldanamycin, gentamicin, glycopeptides, grepafloxacin, herbimycin, imipenem or cilastatin, isoniazid, kanamycin, levofloxacin, lincomycin, lincosamides, linezolid, lipopeptide, lomefloxacin, loracarbef, macrolides, mafenide, meropenem, methicillin, metronidazole, mezlocillin, minocycline, monobactams, moxifloxacin, mupirocin, nafcillin, nalidixic acid, neomycin, netilmicin, nitrofurans, nitrofurantoin, norfloxacin, ofloxacin, oxacillin, oxytetracycline, paromomycin, penicillin G, penicillin V, piperacillin, platensimycin, polymyxin B, pyrazinamide, quinolones, quinupristin/dalfopristin, rifabutin, rifampicin or rifampin, rifapentine, rifaximin, roxithromycin, silver sulfadiazine, sparfloxacin, spectinomycin, spiramycin, streptomycin, sulfacetamide, sulfadiazine, sulfamethizole, sulfamethoxazole, sulfanilamide, sulfasalazine, sulfisoxazole, sulfonamidochrysoidine, teicoplanin, telavancin, telithromycin, temafloxacin, temocillin, tetracycline, thiamphenicol, ticarcillin, tigecycline, tinidazole, tobramycin, trimethoprm, trimethoprim-sulfamethoxazole (co-trimoxazole) (TMP-SMX), and troleandomycin, trovafloxacin, or vancomycin.

In certain embodiments of the methods as disclosed herein, the tissue is contacted with or rinsed in an antimycotics rinse. The antimycotic can include, but is not limited to, the antimycotics: abafungin, albaconazole, amorolfin, amphotericin B, anidulafungin, bifonazole, butenafine, butoconazole, caspofungin, clotrimazole, econazole, fenticonazole, fluconazole, isavuconazole, isoconazole, itraconazole, ketoconazole, micafungin, miconazole, naftifine, nystatin, omoconazole, oxiconazole, posaconazole, ravuconazole, sertaconazole, sulconazole, terbinafine, terconazole, tioconazole, voriconazole, or other agents or compounds with one or more anti-fungal characteristics.

In certain embodiments of the methods as disclosed herein, the tissue is contacted with or rinsed to both antibiotics and antimycotics.

In certain embodiments of the methods disclosed herein, wherein the tissue sample is one or more of placental tissue, amniotic membrane, chorionic membrane, or umbilical cord tissue. In some embodiments, the tissue sample is minced into pieces from about 0.10 cm² to about 1.0 cm²in size. In an embodiment, the tissue sample is minced into pieces from about 0.2 cm² to about 0.50 cm²in size. Before processing the tissue into a particulated tissue suspension, the tissue is cut into smaller pieces, for example, by mincing. Mincing can be performed using any number of methods (e.g., scalpel, scissors or a rotary blade). The minced tissue particles may be square, rounded, oblong or irregular in shape. In certain embodiments, a rotary blade is used to mince the tissue. In some embodiments, the minced tissue comprises tissue having particle sizes ranging from about 0.10 cm² to about 1.0 cm²in size. In certain embodiments, the tissue is minced into pieces from about 0.20 cm² to about 0.50 cm²in size. In general, the tissue is minced into pieces that can be collected using, for example, a 50 mL or 100 mL serological pipette.

In certain embodiments of the methods disclosed herein, the minced tissue and the isotonic solution or the intermediary material are milled in the rotor mill at about 3,000 rpm to about 18,000 rpm. In an embodiment, the minced tissue and the isotonic solution or the intermediary material are milled in the rotor mill at about 6,000 rpm. In some embodiments, the methods as disclosed herein can employ a rotor mill in which a toothed rotor is combined with a sieve of changeable pore size. In certain embodiments, the toothed rotor can spin at a rate of about 1,000 rpm to about 18,000 rpm, such that when a material is loaded into the instrument and the rotor is turned on, centrifugal force propels the material into the sieve and forces it through the sieve by the toothed rotor using cutting and shear forces. In some embodiments, the toothed rotor can spin at a rate of about 1,000 rpm to about 15,000 rpm, about 3,000 rpm to about 12,000 rpm, about 4,000 rpm to about 10,000 rpm, about 5,000 rpm to about 8,000 rpm, about 5,500 rpm to about 7,500 rpm, or about 6,000 rpm to about 7,000 rpm. In an embodiment, the toothed rotor can spin at a rate of about 6,000 rpm.

In certain embodiments, the minced tissue is added to an isotonic solution in a processing chamber. As used herein, the term “solution” refers to a homogeneous mixture composed of two or more substances, and a solution can be liquid or solid (i.e., frozen). As used herein, the term “isotonic solution” refers to when two solutions, separated by a semipermeable membrane, have approximately equal concentrations of solutes and water (i.e., approximately the same osmolarity). In some embodiments, an isotonic solution comprises a solution that is about 270 osm/L to about 320 osm/L. In certain embodiments, the isotonic solution is sterile (i.e., clean and free from bacteria or other microorganisms). In some embodiments, the isotonic solution comprises a frozen isotonic solution (e.g., about 0° C. to about −196° C.). In some embodiments, the isotonic solution can be room temperature (e.g., about 20° C. to about 25° C.). In some embodiments, the isotonic solution can be any temperature ranging from about −200° C. to about 37° C. In some embodiments of the methods disclosed herein, the isotonic solutions can include, but are not limited to, Dulbecco's Modified Eagle's Medium, saline, Plasma-Lyte®-148, Plasma-Lyte®-A, 0.9% NaCl, Phosphate Buffered Saline, Dulbecco's Phosphate Buffered Saline, Earles Balanced Salt Solution, Hank's Balanced Salt Solution, Iscove's Modified Dulbecco's Medium, Alpha Modified Eagle's Medium, Ringer's Lactate solution, Modified Eagle's Medium, Dulbecco's Modified Eagle's Medium/F12, HAM's F-12, or RPMI-1640. In some embodiments, the isotonic solution is frozen. In certain embodiments of the methods disclosed herein, the isotonic solution is frozen into a frozen isotonic solution. In some embodiments, the isotonic solution is frozen into pieces about 0.30 cm² to about 2.5 cm²in size (i.e., a volume of about 0.3 mL to about 2.5 mL). In certain embodiments, the isotonic solution is frozen into pieces about 0.50 cm² to about 1.0 cm²in size (i.e., a volume of about 0.5 mL to about 1.0 mL). In an embodiment, the isotonic solution is frozen into pieces about 2.0 cm²in size (i.e., a volume of about 2.0 mL). In certain embodiments, the processing chamber is first loaded with a confluent layer of the isotonic solution, and then the minced tissue is added on top of the sterile ice in the processing chamber. In some embodiments, an equal volume of the minced tissue is added on top of the sterile ice in the processing chamber.

In certain embodiments, the minced tissue is added to sugar crystals or any other intermediary material that does not exert substantial osmotic forces when dissolved in solution within the processing chamber. In some embodiments, the composition of the sugar crystals may include, but are not limited to, glucose/dextrose, fructose, and galactose. In some embodiments, the sugar crystals are first loaded into the processing chamber prior to adding minced tissue and running the instrument, following which, a solution such as DMEM, for example, could then be added to dissolve the sugar. For example, 1.75 grams sugar crystals, such as glucose, could be added prior to loading minced tissue and running the instrument. Following this, 500 mL of low-glucose DMEM (1 g/L), could be added to result in processed tissue suspended in high-glucose DMEM (4.5 g/L). In certain embodiments, the intermediary material may be comprised of a hydrolysable polymer material. In some embodiments, the formulation of the hydrolysable polymer may include, but is not limited to, polyesters, polyamides, polyanhydrides, polyethers, polyurethanes, polycarbonates, and polyureas. In some embodiments, the material can range in size of about 0.30 cm² to about 2.5 cm² (i.e., a volume of about 0.3 mL to about 2.5 mL). In certain embodiments, the can be about 0.50 cm² to about 1.0 cm²in size (i.e., a volume of about 0.5 mL to about 1.0 mL). In an embodiment, the material is about 2.0 cm²in size (i.e., a volume of about 2.0 mL). In certain embodiments, the processing chamber is first loaded with a confluent layer of the water dissolvable material, and then minced tissue is added on top of the material in the processing chamber.

In certain embodiments, the processing chamber is rotor mill. Exemplary rotor mills can include, but are not limited to, Retsch ZM 200 Ultra Centrifugal Mill, Retsch SR300 Rotor Beater Mill, Fritsch Pulverisette 14, Glatt Rotor Sieve GSE. For these instruments, the final particulate size of the processed tissue is determined by the open porosity of the sieves, with different sieves creating different final particulate sizes. In certain embodiments of the methods disclosed herein, the processing chamber is a rotor mill, and the rotor mill comprises a sieve with a pore size between about 200 μm and about 1 mm. In an embodiment, the pore size of the sieve is about 500 μm. In certain embodiments, the sieve porosity sizes can include, but are not limited to 80 μm, 120 μm, 200 μm, 250 μm, 500 μm, 750 μm, and 1 mm pore sizes. In some embodiments of the methods as disclosed herein, the minced tissue and isotonic solution are milled in the rotor mill at about 3,000 rpm to about 18,000 rpm. In some embodiments, the toothed rotor can spin at a rate of about 1,000 rpm to about 15,000 rpm, about 3,000 rpm to about 12,000 rpm, about 4,000 rpm to about 10,000 rpm, about 5,000 rpm to about 8,000 rpm, about 5,500 rpm to about 7,500 rpm, or about 6,000 rpm to about 7,000 rpm. In an embodiment, the toothed rotor can spin at a rate of about 6,000 rpm. In certain embodiments, the minced tissue and isotonic solution are milled in the rotor mill at about 6,000 rpm. In some embodiments of the methods as disclosed herein, the minced tissue and isotonic solution or other intermediary material are milled in the rotor mill for about 5 seconds to about 60 seconds, about 5 seconds to about 45 seconds, about 10 seconds to about 30 seconds, or about 10 seconds to about 20 seconds. In certain embodiments, the minced tissue and isotonic solution are milled in the rotor mill for about 10 seconds.

Currently available rotor mill instruments are limited by a gap between the sieve and the teeth of the rotor, meaning that soft, and thin materials are not able to be processed in the mill without further modification. To process thin, membranous materials, they must be frozen or mixed with dry ice prior to processing within the rotor mill, negatively influencing both cellular viability and protein integrity. Thus, it is not possible to process said materials within the aforementioned sieve based mills without further modification. The methods disclosed herein provide an intermediary material (e.g. a frozen isotonic solution) that can be used to dose the gap and purge the material to be processed through the sieve without negatively influencing cellular viability or protein integrity. The processed material is then collected from the collection bucket and may be further processed (centrifugation, washing, etc.). In some embodiments of the methods disclosed herein, the isotonic solutions that may be used for this purpose include: Dulbecco's Modified Eagle's Medium, saline, Plasma-Lyte®-148, Plasma-Lyte®-A, 0.9% NaCl, Phosphate Buffered Saline, Dulbecco's Phosphate Buffered Saline, Earles Balanced Salt Solution, Hank's Balanced Salt Solution, Iscove's Modified Dulbecco's Medium, Alpha Modified Eagle's Medium, Ringer's Lactate solution, Modified Eagle's Medium, Dulbecco's Modified Eagle's Medium/F12, HAM's F-12, or RPMI-1640.

In addition to serving as an intermediate material for purging the processed tissue through the sieve, using a frozen or chilled isotonic solution or intermediary material also serves to keep tissue at a cool temperature preventing thermal cycling via the warming and cooling of tissue. During processing, the frozen or chilled isotonic solution or intermediary material also serves to greatly mitigate the generation of heat within tissues due to friction. Pre-loading frozen or chilled isotonic solutions or intermediary materials in the processing chamber (prior to running the instrument) can also serve as a wedge between the sieve and the rotor teeth slowing down rotor acceleration and resulting in more gentle tissue processing.

In certain embodiments of the methods disclosed herein, comprise purging the processing chamber with the isotonic solution to collect residual particulated tissue. In some embodiments, the methods further comprise purging the processing chamber with an additional isotonic solution to collect residual particulated tissue, wherein the additional isotonic solution is a different isotonic solution than the isotonic solution used in step (c).

The methods described herein result in a processed tissue that has been minimally manipulated, avoiding substantial morphological changes to cells, growth factors, and extracellular matrix proteins. Thus, the viability of the cells within the processed tissue is significantly higher than other known methods. For example, the viability of the cells within the processed tissue can be about 20-100%, or about 25-35%, or about 35-45%, or about 45-55%, or about 55-65%, or about 65-75%, or about 75-80%, or about 80-85%, or about 85-90% or about 90-95%, or more than about 95%. In certain embodiments, absolute cell viability ranges between about 20% to about 90% in the processed tissues. Cell viability may be measured using techniques well known in the art. Absolute tissue viability may be determined by comparing viability of processed tissues to non-processed tissues for equivalent tissue weight. If using a fluorescence based technique, the equation ([RFU of 40 mg processed tissue−RFU of negative control]/[RFU of 40 mg non-processed tissue−RFU of negative control])*100% may be used. Alternatively, comparative viability analysis may be performed to determine relative viability comparing different processing methods for equivalent tissue weight. As an example, if using a fluorescence based assay, the equation ([RFU of 40 mg Method 1 processed tissue−RFU of negative control]/[RFU of 40 mg Method 2 processed tissue−RFU of negative control])*100% may be used. Cell viability may be measured by staining the cells with various dyes (e.g., an AlamarBlue™ viability assay). Tools for measuring cell proliferation include probes for analyzing the average DNA content and cellular metabolism in a population, as well as single-cell indicators of DNA synthesis and cell cycle-specific proteins. In some embodiments, the cells can be grown through one to ten or more passages, and the number of cells assessed at predetermined time points to assess the rapidity of cell proliferation and of population growth.

In some embodiments, the processed tissue can be stored by cryopreservation. Cryopreservation refers to a process where organelles, cells, tissues, extracellular matrix, organs, or any other biological constructs susceptible to damage caused by unregulated chemical kinetics are preserved by cooling to very low temperatures (typically around −80° C. to about −196° C.). The temperature range of cryopreservation can be from about −20° C. to about −196° C., or from about −80° C. to about −196° C. For example, the particulated tissue can be re-suspended in a cryopreservation media. For example, the particulated tissue can be cryopreserved in solutions that can include, but are not limited to, Dulbecco's Modified Eagle's Medium, Plasma-Lyte®-148, Plasma-Lyte®-A, 0.9% NaCl, Phosphate Buffered Saline, Dulbecco's Phosphate Buffered Saline, Earles Balanced Salt Solution, Hank's Balanced Salt Solution, Iscove's Modified Dulbecco's Medium, Alpha Modified Eagle's Medium, Ringer's Lactate solution, Modified Eagle's Medium, Dulbecco's Modified Eagle's Medium/F12, HAM's F-12, or RPMI-1640. In some embodiments, cryoprotective agents can be added to the cryopreservation solution. For example, cryoprotective agents can include, but are not limited to, glycerol, dimethyl sulfoxide (DMSO), polyvinylpyrrolidone (PVP), methylcellulose, pentaisomaltose, pentastarch, Dextran-40, trehalose, or sucrose. In certain embodiments, serological components may also be included in the cryopreservation solution. Serological components can include, but are not limited to human serum albumin (powder or liquid form, recombinant or blood derived), platelet rich plasma, insulin, transferrin, or sodium selenite. For example, in an embodiment, pentaisomaltose could be dissolved into a solution (at a w/v concentration of 16%) containing 5% dimethyl sulfoxide (DMSO), and 2.5% human serum albumin Plasma-Lyte®-148 multiple electrolyte solution. In another embodiment, the particulated tissue could be cryopreserved in a solution comprising RPMI-1640 containing 10% DMSO, and 6.5% human serum albumin. In another embodiment, the cryopreservation solution may comprise of 0.9% sodium chloride (NaCl) with 10% DMSO and containing 10% human serum albumin. Tissue suspended in a cryopreservation solution would then be loaded into either into cryopreservation tubes or syringes and appropriately sealed. The syringes or tubes, containing the particulated tissue in the cryopreservation solution, would then undergo a controlled rate freezing within a controlled rate freezer at a rate of −1° C. per minute until its final temperature reaches−80° C. Syringes or tubes would then be packaged and stored at −80° C.

In another aspect, the disclosure provides compositions comprising the particulated tissue prepared by any of the methods disclosed herein.

In yet another aspect, the disclosure provides a kit comprising the composition as disclosed herein in a pharmaceutically acceptable carrier, and a label or instructions for administration of the composition to treat a subject.

In another aspect, the disclosure provides methods of treating a disorder and/or a disease in a subject, wherein the method comprises administering to the subject a therapeutically effective amount of any the compositions as disclosed herein.

In some embodiments, the disorder and/or disease is selected from the group consisting of: an ocular disease (for example, refractive errors, macular degeneration, age-related macular degeneration, cataracts, or uveitis), wound healing (for example, following a surgery, a burn, a trauma or tissue damage), arthritis (for example, osteoarthritis, inflammatory arthritis, rheumatoid arthritis, ankylosing spondylitis, psoriatic arthritis, lupus and/or degeneration and/or inflammation of connective tissues such as cartilage, articular cartilage defect, meniscal damage, osteochondritis dissecans, or aseptic necrosis), an immune-related disease, graft-versus-host disease, and an angiogenesis-related disease.

Furthermore, provided herein are methods of regenerating or repairing a tissue and/or an organ in a subject in need thereof, wherein the methods comprise administering to the subject a therapeutically effective amount of any the compositions as disclosed herein to regenerate or repair the tissue or organ in the subject. In some embodiments, the tissue or organ may be part of the subject's skin, joints, bones, cartilage, respiratory tract, gastrointestinal tract, cardiovascular system, liver, pancreas, bone marrow, or central nervous system. In certain embodiments, administration of the therapeutically effective amount of any the compositions may be topical, transdermal, mucosal, sub-mucosal, muscular, sub-muscular, by inhalation, parenteral, or intravenous administration.

As used herein, the terms “subject” or “patient” to be treated refer to an animal, preferably to a mammal, even more preferably to a human, including an adult, a child, and a human at the prenatal stage. In some embodiments, a subject can refer to a non-human mammal. In certain embodiments, a subject is a human subject.

As used herein, the term “treatment”, “treat” or “treating” refers to any act intended to ameliorate the health status of patients such as therapy, prevention, prophylaxis and retardation of the disorder and/or disease. In certain embodiments, such term refers to the amelioration or eradication of a disease or symptoms associated with a disease as disclosed herein. In other embodiments, this term refers to minimizing the spread or worsening of the disease resulting from the administration of one or more therapeutic agents to a subject with such a disease.

The term “therapeutically effective amount” refers to an amount of any of the compositions as disclosed herein that is administered to a subject that is sufficient to constitute a treatment of the disorder and/or disease as disclosed above. In some embodiments, this term may refer to the amount of the composition necessary to repair and/or regenerate the tissue(s) or organ(s) of the subject being treated.

The compositions for use in the methods as disclosed herein are typically “pharmaceutically acceptable”. The term “pharmaceutically acceptable” as used herein refers to compositions comprising various components, such as, for example, carriers, diluents, preservatives, and/or cryopreservatives, and such components are typically suitable for administration to a human subject, as well as to a non-human mammalian subject.

In certain embodiments, the compositions can be administered one or more times in order to effectively treat the subject. A decision as to how many doses (and when to administer the dose(s)) will typically be made by an individual who is supervising the subject's treatment, such as a treating physician, doctor, veterinarian, or nurse. A combination of factors will typically be taken into account in making such a decision. For example, factors taken in to account may include a timescale of appropriateness as assessed on the basis of past experience, either with the individual subject patient's condition or others with similar conditions, or on the degree of debility of the subject, or on the degree of pain experienced by the subject or may be based on a test independent of the subject's own assessment.

### EXAMPLES

### Example 1. Exemplary Methods for Processing Tissue

### Steps in One Iteration:

1. Tissue is bluntly minced using a scalpel or modified array of circular fabric cutting blades spaced.
2. Tissue is collected using automated pipette.
3. Sterile Ice is loaded into the edge of the rotor, interfacing the sieve.
4. Minced Tissue is dispensed on top of the ice chips close to the surface of the sieve.
5. The instrument is dosed and then ran at the minimum rotor speed available.
6. To purge any leftover tissue/ice left in the sieve, isotonic fluid is poured into the top of the instrument and collected in the collection bucket.
7. Further processing steps (e.g. washing, rinsing, centrifugation prior to cryopreservation).

### Steps in Another Iteration:

Pre-Processing Prep

| Day 1 | Day 1 Prepare Processing Solutions |
|---|---|
| 1a | Post-Processing Solution Mixture Containing Antibiotics |
| | and Antimycotics (comprising glucose in HEPES |
| | buffer containing antibiotics/antifungals, Human Serum |
| | Albumin or Recombinant Albumin and Insulin Transferrin |
| | and Sodium Selenite Additive [ITSE]) |
| 1b | Cold Plasma-Lyte ® 148/Saline or PBS |
| 1c | Cold ACK Lysing Buffer |
| 1d | Cold DMEM (25 mM HEPES, 4.5 g/L Glucose) |
| 1e | Make Frozen Isotonic Solution |
| Day 2 | Day 2 Prepare Processing Solutions |
| 2a | Cryopreservative Solution (comprising Human Serum |
| | Albumin [Blood Derived or Recombinant], DMSO, Penta- |
| | isomaltose, in Plasma-Lyte ® 148/Plasma-Lyte ® A) |
| 2b | Saline/PBS or Plasma-Lyte ®148 (PL148 or PBS preferred) |
| Day 1 | Sterilize All Instruments and Supplies |
| | Cutting tools, rotary blades and Forceps, etc. |
| | Metal Pans for Amnion Handling Steps |
| | Cutting Boards |
| | Metal Spatulas/Spoons |
| | Ice Trays |
| | 100 mL Serological Pipettes |
| | 150 mL, 500 mL Containers (500 mL containers may need |
| | permeable membrane) |

Day 1

| | |
|---|---|
| Step 1 | Open up Amnion/Chorion Container under cleanroom |
| Step 2 | Separate Amnion from Chorion in 500 mL Cold PBS (4° C.) |
| Step 3 | Transfer Amnion to Metal Pan Containing Cold DMEM |
| Step 4 | Transfer Bloody Chorion to Sterile Metal Pan containing |
| | Sterile ACK Lysing Buffer |
| Step 5 | Gently rub surface of bloody chorion surfaces and gently |
| | remove clots. For small, persistent clots, use cell scraper to |
| | gently remove clots from surface, repeat up to 2X with fresh |
| | ACK lysing buffer |
| Step 6 | Place Chorion in Pan Containing DMEM along with Amnion |
| Step 7 | Place Amnion and Chorion together on Cutting Board and |
| | Roughly Mince using sterile rotary blade array as finely as |
| | possible |
| Step 8 | Pick up Minced Amnion Using Autopipettor with 50 mL |
| | (100 mL preferred) Serological Pipette and Note Rough |
| | Tissue Volume. Place into a pre-weighed 150 mL container. |
| Step 9 | Weigh container containing tissue and then add equivalent |
| | amount of cold DMEM to match initial tissue volume |
| Step 10 | Load enough cubed frozen isotonic solution to create one |
| | confluent layer of the frozen isotonic solution within |
| | assembled rotor mill [note how many ice cubes are |
| | present to approximate fluid volume] (make sure it is |
| | assembled with 500 μm sieve) |
| Step 11 | Load 1/2 of total volume into rotor mill on top of the frozen |
| | isotonic solution as close to the sieve as possible using auto |
| | pipettor |
| Step 12 | Put rotor mill cassette lid on top of 900 mL cassette collector |
| | and close instrument |
| Step 13 | Make sure rotor mill is set at 6,000 rpm and run the rotor mill |
| | for 10 seconds |
| Step 14 | Add cubes of the frozen isotonic solution into instrument from |
| | top to purge sieve. Note total volume added. |
| Step 15 | Add 50 mL of cold DMEM from top of instrument to finish |
| | purging tissue and frozen isotonic solution from sieve |
| Step 16 | Open instrument and add enough post-processing solution |
| | containing antibiotics and antimycotics to match total tissue |
| | and fluid volume added during processing. |
| Step 17 | Use autopipettor to collect total volume into a 500 mL or |
| | 1,000 mL container |
| Step 18 | Place into tissue culture incubator (37° C., 5% CO₂) for 12-18 |
| | hours for antibiotics/antifungal treatment |
| Step 19 | Repeat Steps 10-18 for 2nd half of amniotic/chorionic tissue |

Day 2

| | |
|---|---|
| Step 1 | Take tissue out from incubator and place into four pre- |
| | weighed centrifuge tubes at equal volumes (divide by four) |
| Step 2 | Centrifuge for 5 minutes |
| Step 3 | Remove fluid Supernatant from tube. It may be necessary to |
| | use a spatula/spoon in order to remove tissue floating at top |
| | of tube and transfer tissue to a new tube so that vacuum |
| | aspiration can be successfully performed on tissue pellet |
| Step 4 | Determine total tissue weight, and re-suspend in 200 mL |
| | of Plasma-Lyte ®-148 per centrifuge tube, close containers, |
| | and tip containers up and down to distribute tissue |
| | in fluid to rinse |
| Step 5 | Centrifuge for 5 minutes |
| Step 6 | Remove fluid Supernatant from tube. It may be necessary |
| | to use a spatula/spoon in order to remove tissue floating |
| | at top of tube and transfer tissue to a new tube so that |
| | vacuum aspiration can be successfully performed on tissue |
| | pellet—make sure to note total tissue weight after |
| | removing supernatant |
| Step 7 | Resuspend tissue in cryopreservative—Keep solution and |
| | fluid cold (use refrigeration or ice bath to keep cold |
| | while dispensing into syringes). |
| Step 8 | Load solution into each syringe using autopipettor, |
| | etc.—Keep syringes cool while loading (use ice or |
| | refrigeration while loading). |
| Step 9 | Seal syringes (add on plunger with stopper, etc.). |
| Step 10 | Place sealed syringes into cryopreservation rack. |
| Step 11 | Place into controlled rate freezer and use a protocol in which |
| | vials are chilled at a rate of −1° C./minute until temperature |
| | reaches −80° C. |
| Step 12 | Package, etc. |

### Example 2

The goal of this experiment was to elucidate the best methodology for utilizing the rotor mill such that the highest tissue viability of particulated amniotic membrane is achieved. Various methods for loading the intermediary isotonic solution were tested. Condition 1: Running rotor mill first and pouring minced tissue at the same time as a frozen isotonic solution. Condition 2: Loading frozen isotonic solution into the rotor mill and adding minced tissue on top prior to running the rotor mill. Condition 3: Running rotor mill first and pouring minced tissue suspended in DMEM first from the top, followed by adding in ice afterwards from the top to purge the sieve). The results demonstrate that based on AlamarBlue™ viability assays, it is more advantageous to viability to pre-load the instrument with frozen isotonic solution prior to running it.

Amnion and chorion were received in 0.9% NaCl and packaged on wet ice. Amnion and chorion were placed into PBS solution at 4° C. Amnion was peeled from the chorion and placed into a metal tray containing 500 mL DMEM. Amnion was removed and placed onto cutting board, following which, it was minced using an array of rotary cutting blades. After this, tissue was picked up using an automated pipettor and split into three separate conditions of equal volumes (1. Condition 1, 2. Condition 2, 3. Condition 3). All conditions were processed in the rotor mill at a speed of 6,000 rpm for 10 seconds. After processing in the rotor mill, collected particulated tissues were aliquoted into multi-well tissue culture plates within cell culture media such that each well contained the same amount of collected particulated tissue. AlamarBlue™ reagent was added to the samples such that the end concentration of AlamarBlue™ was 10%. Complete AlamarBlue™ reagent media without tissue added served as a negative control. Tissue culture plates were then placed into a humidifying cell culture incubator at 37° C., 5% CO₂ for at least 12 hours. After at least 12 hours, plates were removed from the incubator and 100 μL aliquots of the reduced AlamarBlue™ media supernatants were taken from each sample and placed into 96 well plates. Fluorescence intensity was measured using a Synergy H1 plate reader at 560 nm excitation and 590 nm emission. The results show the greatest cell viability when the rotor mill was pre-loaded with frozen isotonic solution prior to running.

### Example 3

The goal of this experiment was to test the simultaneous processing of amnion and chorion together using the method of pre-loading the rotor mill with the frozen isotonic solution while also testing how injection of particulated tissue under different conditions can influence viability. Particulated samples processed using a frozen isotonic solution pre-loaded in the rotor mill (outfitted with a 500 μm diameter sieve) were injected through either a 22 gauge needle or a 21 gauge needle. The results demonstrate that needle size can influence post-injection viability. The results suggest that the utilization of thin-walled needles can improve flowability and prevent tissue from getting stuck in needle during injection. It was also determined that when processing the amnion and chorion together, the 500 μm sieves do not negatively impact cell viability.

Amnion and chorion were received in 0.9% NaCl and packaged on wet ice. Amnion and chorion were placed into cold PBS (4° C.) solution. Amnion and chorion together were placed into a metal tray containing 500 mL DMEM. Tissue was removed from metal tray and placed onto cutting board, following which, it was minced using an array of rotary cutting blades. After this, tissue was picked up using an automated pipettor and processed using standard methodology (DMEM frozen into cubes approximately 2 cc pre-loaded in to the rotor mill, and running the rotor mill for 10 seconds at 6,000 rpm). Particulated tissue was re-suspended into complete cell culture media. 3 mL BD Luer Lock Syringes were loaded with 2 mL of particulated tissue and injected into multi-well tissue culture plates using 22 gauge or 21 gauge needles. AlamarBlue™ reagent was added to the samples such that the end concentration of AlamarBlue™ was 10%. Complete AlamarBlue™ reagent media without tissue added served as a negative control. Tissue culture plates were then placed into a humidifying cell culture incubator at 37° C., 5% CO₂ for at least 12 hours. After at least 12 hours, plates were removed from the incubator and 100 μL aliquots of the reduced AlamarBlue™ media supernatants were taken from each sample and placed into 96 well plates. Fluorescence intensity was measured using a Synergy H1 plate reader at 560 nm excitation and 590 nm emission. The results show the greatest cell viability when the particulated tissue was injected using a needle with a larger circumference (i.e. a lower gauge).

1. A method of producing a particulated tissue, the method comprising:
(a) obtaining a tissue sample;
(b) mincing the tissue sample to produce a minced tissue;
(c) loading an isotonic solution into a processing chamber;
(d) adding the minced tissue to the isotonic solution in the processing chamber;
(e) milling the minced tissue in the processing chamber into the particulated tissue; and
(f) collecting the particulated tissue.

2. A method of producing a particulated tissue, the method comprising:
(a) obtaining a tissue sample;
(b) mincing the tissue sample to produce a minced tissue;
(c) loading an intermediary material into a processing chamber;
(d) adding the minced tissue to the intermediary material in the processing chamber;
(e) milling the minced tissue in the processing chamber into the particulated tissue; and
(f) collecting the particulated tissue.

3. The method of claim 1, wherein the tissue sample is one or more of placental tissue, amniotic membrane, chorionic membrane, or umbilical cord tissue.

4. The method of claim 1, wherein the tissue sample is minced into pieces from about 0.10 cm² to about 1.0 cm²in size.

5. The method of claim 4, wherein the tissue sample is minced into pieces from about 0.2 cm² to about 0.50 cm²in size.

6. The method of claim 1, wherein the isotonic solution is frozen into a frozen isotonic solution.

7. The method of claim 6, wherein the frozen isotonic solution is about 0.30 cm² to about 2.5 cm²in size.

8. The method of claim 1, wherein the processing chamber is a rotor mill, and the rotor mill comprises a sieve with a pore size between about 200 μm and about 1 mm.

9. The method of claim 8, wherein the pore size of the sieve is about 500 sm.

10. The method of claim 1, wherein the minced tissue and the isotonic solution or the intermediary material are milled in the rotor mill at about 3,000 rpm to about 18,000 rpm.

11. The method of claim 10, wherein the wherein the minced tissue and the isotonic solution or the intermediary material are milled in the rotor mill at about 6,000 rpm.

12. The method of claim 1, wherein the minced tissue and the isotonic solution or the intermediary material are milled in the rotor mill for about 5 seconds to about 60 seconds.

13. The method of claim 12, wherein the minced tissue and the isotonic solution or the intermediary material are milled in the rotor mill for about 10 seconds.

14. The method of claim 1, comprising purging the processing chamber with the isotonic solution to collect residual particulated tissue.

15. The method of claim 14, further comprising purging the processing chamber with an additional isotonic solution to collect residual particulated tissue, wherein the additional isotonic solution is a different isotonic solution than the isotonic solution used in claim 1.

16. A composition comprising the particulated tissue of claim 1.

17. A kit comprising: the composition of claim 16 in a pharmaceutically acceptable carrier, and a label or instructions for administration of the composition to treat a subject.

18. A method of treating a disorder in a subject, the method comprising administering to the subject a therapeutically effective amount of the composition of claim 16.

19. The method of claim 18, wherein the disorder is selected from the group consisting of: an ocular disease (for example, refractive errors, macular degeneration, age-related macular degeneration, cataracts, or uveitis), wound healing (for example, following a surgery, a burn, a trauma or tissue damage), arthritis (for example, osteoarthritis, inflammatory arthritis, rheumatoid arthritis, ankylosing spondylitis, psoriatic arthritis, lupus and/or degeneration and/or inflammation of connective tissues such as cartilage, articular cartilage defect, meniscal damage, osteochondritis dissecans, or aseptic necrosis), an immune-related disease, graft-versus-host disease, and an angiogenesis-related disease.

20. The method of claim 18, wherein administration of the therapeutically effective amount of any the compositions may be topical, transdermal, mucosal, sub-mucosal, muscular, sub-muscular, by inhalation, parenteral, or intravenous administration.

21. The method of claim 18, wherein the subject is a human (for example, an adult, a child, or a human at the prenatal stage) or a non-human mammal.
